(19) United States
(12) Patent Application Publication
(10) Pub. No.: US 2026/0124413 A1
(43) Pub. Date: May 7, 2026

(54) BREATHING SYSTEM WITH INTEGRATED IDENTITY VERIFICATION

(71) Applicant: Telesair, Inc., Irvine, CA (US)

(72) Inventors: Chi Wai CHOY, Irvine, CA (US); Cheng WANG, Shenzhen (CN)

(73) Assignee: Telesair, Inc., Irvine, CA (US)

(21) Appl. No.: 18/934,517

(22) Filed: Nov. 1, 2024

(51) Int. Cl. A61M 16/00; A61M 16/06; A61M 16/08; A61M 16/10

(52) U.S. Cl. CPC A61M 16/022; A61M 16/0003; A61M 16/06; A61M 16/0875; A61M 16/1075; A61M 2205/3368; A61M 2205/60

(57) ABSTRACT

A breathing system, including a breathing apparatus, a breathing gas supply conduit, and a patient interface for a user; where the breathing apparatus is operatively connected to one end of the breathing gas supply conduit, and the patient interface is operatively connected to the other end of the breathing gas supply conduit; where the breathing apparatus is configured to provide breathing gas to the user through the breathing gas supply conduit and the patient interface; where at least one of the breathing gas supply conduit or the patient interface is equipped with a verification chip, and the verification chip is electrically connected to the breathing apparatus and used for identity verification of either or both the breathing gas supply conduit or the patient interface.

### TECHNICAL FIELD

The present disclosure relates to the technical field of medical devices and, in particular, to a breathing system.

### BACKGROUND

For patients who require the use of respiratory aids, providing a stable and reliable supply of breathing gas is very beneficial. However, as the breathing system is a complex system that requires multiple components to work together, and there are many suppliers providing related components, mixing products from different suppliers may cause incompatibility, which results in the breathing system unable to function properly and even posing a risk to the safety of the lives of the patients.

This background information is provided to reveal information believed by the applicant to be of possible relevance to the present disclosure. No admission is necessarily intended, nor should be construed, that any of the preceding information constitutes prior art against the present disclosure.

### SUMMARY

The present disclosure provides a breathing system. According to the breathing system,

In a first aspect, an embodiment of the present disclosure provides a breathing system, including a breathing apparatus, a breathing gas supply conduit, and a patient interface for a user;
where the breathing apparatus is operatively connected to one end of the breathing gas supply conduit, and the patient interface is operatively connected to the other end of the breathing gas supply conduit;
where the breathing apparatus is configured to provide breathing gas to the user through the breathing gas supply conduit and the patient interface;
where at least one of the breathing gas supply conduit or the patient interface is equipped with a verification chip, and the verification chip is electrically connected to the breathing apparatus and used for identity verification of the at least one of the breathing gas supply conduit or the patient interface.

In a possible implementation, in a case that the breathing gas supply conduit is equipped with the verification chip, the verification chip of the breathing gas supply conduit carries first identity information of the breathing gas supply conduit, and the first identity information is used for the breathing apparatus to perform identity verification on the breathing gas supply conduit.

In a possible implementation, the first identity information is a unique device identification (UDI) of the breathing gas supply conduit.

In a possible implementation, the verification chip of the breathing gas supply conduit is positioned at the one end or the other end of the breathing gas supply conduit.

In a possible implementation, in a case that the patient interface is equipped with the verification chip, the verification chip of the patient interface carries second identity information of the patient interface, and the second identity information is used for the breathing apparatus to perform identity verification on the patient interface.

In a possible implementation, the second identity information is a UDI of the patient interface.

In a possible implementation, the patient interface includes a cannula or a mask on which the verification chip is positioned.

In a possible implementation, the breathing system further includes a temperature sensor electrically connected to the breathing apparatus;
where the temperature sensor is configured to report a temperature within the breathing gas supply conduit to the breathing apparatus;
where the temperature sensor is positioned at the breathing gas supply conduit or the patient interface.

In a possible implementation, in a case that the temperature sensor is positioned at the breathing gas supply conduit, the temperature sensor is positioned to be flushed with or recessed into a wall of the breathing gas supply conduit.

In a possible implementation, in a case that both of the verification chip and the temperature sensor are positioned at the breathing gas supply conduit or the patient interface, the verification chip and the temperature sensor are electrically connected to the breathing apparatus in series.

In a possible implementation, the breathing system further includes a heating element electrically connected to the breathing apparatus;
where the heating element is configured to regulate and maintain a temperature of the breathing gas passing through the breathing gas supply conduit.

In a possible implementation, a circuit for an electrical connection between the heating element and the breathing apparatus and a circuit for an electrical connection between the temperature sensor and the breathing apparatus are set up independently.

In a possible implementation, a circuit for an electrical connection between the heating element and the breathing apparatus and a circuit for an electrical connection between the temperature sensor and the breathing apparatus share one wire used for both of the circuits.

In a second aspect, an embodiment of the present disclosure provides a breathing system, including a breathing apparatus, a breathing gas supply conduit, and a patient interface for a user;
where the breathing apparatus is operatively connected to one end of the breathing gas supply conduit, and the patient interface is operatively connected to the other end of the breathing gas supply conduit;
where the breathing apparatus is configured to provide breathing gas to the user through the breathing gas supply conduit and the patient interface;
where at least one of the breathing gas supply conduit or the patient interface is equipped with a verification chip, and the verification chip is used for identity verification of the at least one of the breathing gas supply conduit or the patient interface.

It should be understood that the content described in this section is not intended to identify the key or important features of the embodiments of the present disclosure, nor to limit the scope of the present disclosure. Other features of the present disclosure will be easily understood through the following description.

### BRIEF DESCRIPTION OF THE DRAWINGS

The drawings are used for a better understanding of the present solution but do not constitute any limitation on the present disclosure. Throughout the drawings, identical or similar reference numbers designate identical or similar elements.

FIG. 1 is a schematic diagram of a breathing system according to an embodiment of the present disclosure.

FIG. 2a is a schematic diagram of a breathing system including a breathing gas supply conduit equipped with a verification chip according to an embodiment of the present disclosure.

FIG. 2b is a schematic diagram of another breathing system including a breathing gas supply conduit equipped with a verification chip according to an embodiment of the present disclosure.

FIG. 3 is a schematic diagram of a breathing system including a patient interface equipped with a verification chip according to an embodiment of the present disclosure.

FIG. 4 is a schematic diagram of a breathing system including a temperature sensor according to an embodiment of the present disclosure.

FIG. 5 is a perspective schematic diagram of a breathing gas supply conduit according to an embodiment of the present disclosure.

FIG. 6a is a schematic diagram of a circuit for a heating element and a temperature sensor according to an embodiment of the present disclosure.

FIG. 6b is a schematic diagram of another circuit for a heating element and a temperature sensor according to an embodiment of the present disclosure.

### DETAILED DESCRIPTION OF THE EMBODIMENTS

In the following description, reference is made to the accompanying figures, which form part of the disclosure, and which show, by way of illustration, specific aspects of embodiments of the present disclosure or specific aspects in which embodiments of the present disclosure may be used. It is understood that embodiments of the present disclosure may be used in other aspects and include structural or logical changes not depicted in the figures. The following detailed description, therefore, is not to be taken in a limiting sense, and the scope of the present disclosure is defined by the appended claims.

The term “include” used herein and its variations are open inclusion, that is, “include but not limited to”. The term “based on” means “at least partly based on”. The term “an embodiment” represents “at least one embodiment”; the term “another embodiment” represents “at least one another embodiment”; and the term “some embodiments” represents “at least some embodiments”. Related definitions of other terms will be provided in the following.

It should be noted that concepts such as “first”, and “second” mentioned in the present disclosure are merely used to distinguish different apparatuses, modules, or units, but not to limit the sequence or interdependency of functions executed by these apparatuses, modules, or units.

It should be noted that the singular or plural modification mentioned in the present disclosure is illustrative and not restrictive, and those skilled in the art should understand that it should be understood as “one or more” unless clearly defined in the context otherwise.

As described in related art, for patients who require the use of respiratory aids, providing a stable and reliable supply of breathing gas is very beneficial. However, as the breathing system is a complex system that requires multiple components to work together, and there are many suppliers providing related components, mixing products from different suppliers may cause incompatibility, which results in the breathing system unable to function properly and even posing a risk to the safety of the lives of the patients.

In order to overcome the above incompatibility issue, embodiments of the present disclosure provide a breathing system, which includes a breathing apparatus, a breathing gas supply conduit, and a patient interface for a user. Among them, the breathing apparatus is operatively connected to one end of the breathing gas supply conduit, and the patient interface is operatively connected to the other end of the breathing gas supply conduit; where the breathing apparatus is configured to provide breathing gas to the user through the breathing gas supply conduit and the patient interface; and at least one of the breathing gas supply conduit or the patient interface is equipped with a verification chip, and the verification chip is used for identity verification of the at least one of the breathing gas supply conduit or the patient interface.

In some cases, the verification chip may be electrically connected to the breathing apparatus, where the identity verification may be achieved based on such electrical connection, that is, information exchange for realizing the identity verification would be conducted in a wired manner. It is also possible that the verification chip is a wireless chip which does not need to be electrically connected to the breathing apparatus, in this way, the identity verification would be done in a wireless manner. Different connection implementations improve the flexibility and diversity of the overall breathing system.

In the following, description will be made by taking the wired connection as an example, but it should be noted that the detailed implementations would also be applicable for the wireless case (where identity verification is performed based on the verification chip in a wireless way), including the arrangements of the verification chip, the working principle of the verification chip, as well as the description related to the temperature sensor, the heating element.

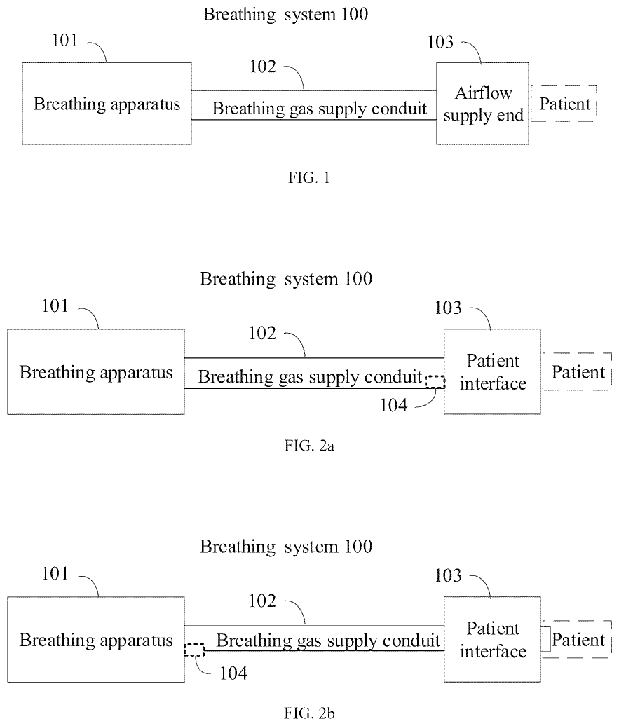

FIG. 1 is an exemplary schematic diagram of a breathing system according to an embodiment of the present disclosure. As shown in FIG. 1, the breathing system 100 includes a breathing apparatus 101, a breathing gas supply conduit 102, and a patient interface 103 for a user.

The user may be a patient requiring breathing gas (which could be oxygen, nitrogen, or any other gas for medical treatment), or may be a medical personnel or a product tester who tests the product capabilities of the breathing system. Hereinafter, no strict distinction is made between the “user” and the “patient”, and their meanings are the similar without causing ambiguity.

In some cases, the breathing apparatus 100 may be a breathing device. In an implementation, the breathing device may be used to provide breathing gas for assisting breathing of a patient. The flow rate of the breathing gas, whether high or low, is not limited in the embodiments of the present application. For example, the breathing device may be used for humidification, for oxygen supply (e.g., for medical treatment), and etc. More specifically, the breathing device may be a Bonhawa Humidifier.

In some cases, the patient interface 103, which is arranged at the user side/patient side, is used to provide the breathing gas to a patient in a contact manner or a non-contact manner. For example, the patient interface 103 may be a cannula, a mask, and etc.

In some cases, the breathing gas supply conduit 102 is used to connect the breathing apparatus 101 and the patient interface 103 for facilitating the flow delivery therebetween, and the breathing gas provided by the breathing apparatus 101 is transmitted to the patient interface 103 through the breathing gas supply conduit 102.

As shown in FIG. 1, the breathing apparatus 101 is operatively connected to one end of the breathing gas supply conduit 102, and the patient interface 103 is operatively connected to the other end of the breathing gas supply conduit 102. The breathing apparatus 101 is configured to provide breathing gas to the user through the breathing gas supply conduit 102 and the patient interface 103.

Among them, at least one of the breathing gas supply conduit 102 or the patient interface 103 is equipped with a verification chip (not shown in FIG. 1), and the verification chip is used for identity verification of the at least one of the breathing gas supply conduit 102 or the patient interface 103. As one possible choice, the verification chip is electrically connected to the breathing apparatus 101.

Either or both of the breathing gas supply conduit 102 and the patient interface 103 could be equipped with a verification chip. The provision of such verification chip is for authentication purpose, when the component (which could be either of the breathing gas supply conduit 102 and the patient interface 103) has a verification chip, its identity could be authenticated based on the verification chip, in this way, the safety of the whole breathing system is ensured, and the compatibility among different components in the breathing system could be improved. There is no limitation with respect to the position for such verification chip, as long as an electrical connection between the verification chip and the component possessing the verification chip is ensured.

With respect to the device which performs the identity verification, it could be a device in the breathing system, i.e., a controller deployed in the breathing system, or it could also be a device which is outside the breathing system but can communicate with the verification chip.

With respect to the way in which the identity verification is done, it can be done automatically or based on some trigger. For example, the verification identity of the component (which could be either of the breathing gas supply conduit 102 and the patient interface 103) may contain identity information of the component therein, when the component is placed in the breathing system or connected to an existing component in the system, the “in-place” state of the newly placed component would be detected by the breathing system (e.g., by a controller therein), then the identity information contained in the component would be automatically delivered to the breathing system, so that the breathing system could complete the identity verification of the component, and if the result of the identity verification shows that the newly placed component is an unauthorized component, then some warning prompt would be triggered. For another example, the identity verification may also be done in a trigger-based way. Upon detecting the “in-place” state of the component, it is also possible for the breathing system to give a notification about such “in-place” state, so that the identity verification could be triggered in a manual manner, e.g., by the user. Here, “unauthorized” means that the component is deem incompatible by the breathing system, for example, there could be a whitelist stored in the breathing system (e.g., inside the controller), so when a component whose identity information provided by the verification chip does not exist on the whitelist, a warning prompt would be issued to notify the user that the component is unauthorized; for another example, the breathing system could also store identity information of the breathing apparatus, and when the identity information of the breathing apparatus is not consistent with the identity information of the newly placed component, such case would also be regarded as unauthorized.

Since breathing gas supply conduits and patient interfaces, which are commonly used consumables in breathing systems, may be produced by various manufacturers, these components can differ in performance, product size, and other product characteristics. If a breathing gas supply conduit from a manufacturer different from a manufacturer of the breathing apparatus is used, it may pose potential safety risks. Therefore, identity verification of the breathing gas supply conduit/patient interface is a valid means to ensure product performance and user safety. Additionally, it should be noted that the consistency verification would be between the breathing apparatus and the breathing gas supply conduit/patient interface with the verification chip, or between one or more of specific units inside the breathing apparatus and the breathing gas supply conduit/patient interface with the verification chip, which is not limited in the embodiments of the present disclosure. This authorization test ensures compatibility among different components in the breathing system, since it is more likely that components from the same manufacturer would have a higher compatibility.

In the following, description will be made by elaborating different arrangements of verification chip(s).

FIG. 2a is a schematic diagram of a breathing system including a breathing gas supply conduit equipped with a verification chip according to an embodiment of the present disclosure. FIG. 2b is a schematic diagram of another breathing system including a breathing gas supply conduit equipped with a verification chip according to an embodiment of the present disclosure.

As shown in FIG. 2a and FIG. 2b, the breathing gas supply conduit 102 is equipped with the verification chip 104. The verification chip 104 of the breathing gas supply conduit 102 carries first identity information of the breathing gas supply conduit 102, and the first identity information is used for the breathing apparatus 101 to perform identity verification on the breathing gas supply conduit 102. Here the first identity information could also be used for identity verification in various ways as described with respect to FIG. 1, which will not be repeated herein for brevity.

In an implementation, the first identity information is a unique device identification (UDI) of the breathing gas supply conduit.

It should be understood that breathing gas supply conduits, which are commonly used consumables in breathing systems, are produced by various manufacturers. Breathing gas supply conduits from different manufacturers may differ in performance and product size. If a breathing gas supply conduit from a manufacturer different from a manufacturer of the breathing apparatus is used, it may pose potential safety risks. Therefore, identity verification of the breathing gas supply conduit is a valid means to ensure product performance and user safety. By verifying the verification chip on the breathing gas supply conduit through the breathing apparatus, the breathing apparatus can determine whether the breathing gas supply conduit is authorized, ensuring that the breathing apparatus and the breathing gas supply conduit either come from the same manufacturer or meet the specifications to ensure compatibility.

In an implementation, the verification chip of the breathing gas supply conduit is positioned at one end or the other end of the conduit.

The verification chip of the breathing gas supply conduit is positioned at one of the two ends of the conduit. On one hand, this simplifies the manufacturing process, and on the other hand, placing the chip at the interface between the breathing gas supply conduit and the breathing apparatus (FIG. 2a) or the patient interface (FIG. 2b) further reduces the complexity of the circuit.

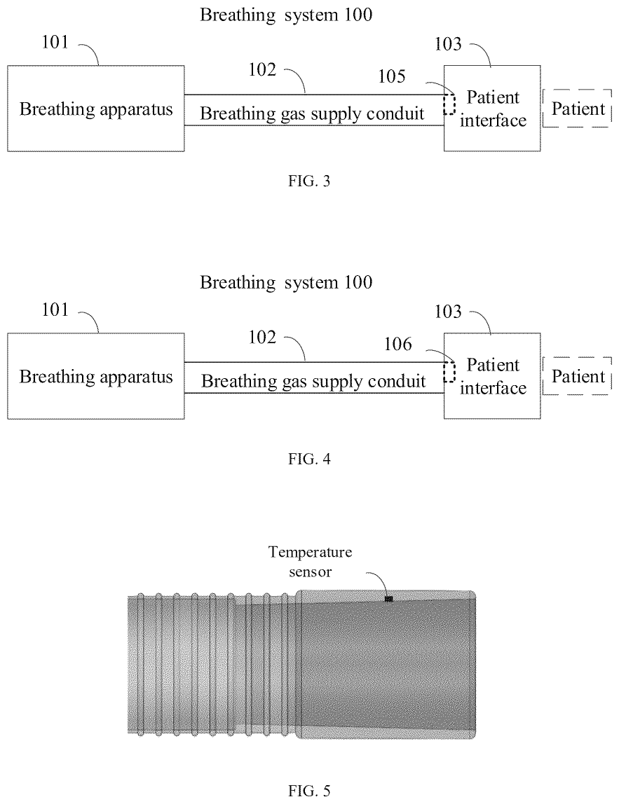

FIG. 3 is a schematic diagram of a breathing system including a patient interface equipped with a verification chip according to an embodiment of the present disclosure. As shown in FIG. 3, the patient interface 103 is equipped with the verification chip 105, the verification chip 105 of the patient interface 103 carries second identity information of the patient interface 103, and the second identity information is used for the breathing apparatus 101 to perform identity verification on the patient interface 103.

In an implementation, the second identity information is a UDI of the patient interface. Here the second identity information could also be used for identity verification in various ways as described with respect to FIG. 1, which will not be repeated herein for brevity. Besides, the UDI of the breathing gas supply conduit and the UDI of the patient interface would be different.

Similar to the breathing gas supply conduit, patient interfaces are also commonly used consumables, produced by more manufacturers and with more diverse product characteristics. As the final component that delivers the breathing gas to a patient, the patient interface would not only conform to ergonomic standards for patient comfort, but more importantly, would function properly with the breathing gas supply conduit and the breathing apparatus. By verifying the verification chip on the patient interface through the breathing apparatus, the breathing apparatus can determine whether the patient interface is authorized, ensuring that the breathing apparatus, the breathing gas supply conduit, and the patient interface either come from the same manufacturer or meet the specifications to ensure compatibility.

In an implementation, the patient interface includes a cannula or a mask on which the verification chip is positioned.

FIG. 4 is a schematic diagram of a breathing system including a temperature sensor, according to an embodiment of the present disclosure. As shown in FIG. 4, the breathing system includes a breathing apparatus 101, a breathing gas supply conduit 102, a patient interface 103, and further includes a temperature sensor 106. And the temperature sensor 106 is electrically connected to the breathing apparatus 101.

Among them, the temperature sensor 106 is configured to report a temperature within the breathing gas supply conduit 102 to the breathing apparatus 101.

Although the above chip is not shown in FIG. 4, in fact, the above arrangements of the verification chip(s) in the previous embodiment are also applicable to this embodiment, that is, the verification chip can be used in combination with the temperature sensor.

In an implementation, the temperature sensor is positioned at the breathing gas supply conduit or the patient interface.

The temperature sensor may be positioned at any part of the breathing gas supply conduit. In some cases, the temperature sensor may be positioned at the other end of the breathing gas supply conduit (that is, the end connected to the patient interface).

It should be understood that positioning the temperature sensor close to the user can facilitate obtaining the perceived temperature of the user.

In an implementation, when the temperature sensor is positioned at the breathing gas supply conduit, as shown in FIG. 5, the temperature sensor is positioned to be flushed with or recessed into a wall of the breathing gas supply conduit.

This approach offers several benefits: it simplifies the manufacturing process of the breathing gas supply conduit, reduces the resistance in the flow path, and minimizes the internal surface area that contributes to cooling the breathing gas.

In an implementation, when both the verification chip and the temperature sensor are positioned at the breathing gas supply conduit or the patient interface, the verification chip and the temperature sensor are electrically connected to the breathing apparatus in series.

Specifically, when both the verification chip and the temperature sensor are positioned at the breathing gas supply conduit, the verification chip and the temperature sensor are electrically connected to the breathing apparatus in series. For example, as shown in FIG. 2a, the temperature sensor is connected in series with the chip 104 at or near the position indicated. In this case, the breathing apparatus performs identity verification on the breathing gas supply conduit using the verification chip, while the temperature sensor reports the temperature near the interface between the breathing gas supply conduit and the patient interface.

And when both the verification chip and the temperature sensor are positioned at the patient interface, the verification chip and the temperature sensor are electrically connected to the breathing apparatus in series. For example, this scenario can be referred to in FIG. 3, the temperature sensor is connected in series with the chip 105 at or near the position indicated. In this case, the breathing apparatus performs identity verification on the patient interface using the verification chip, while the temperature sensor reports the temperature near the interface between the breathing gas supply conduit and the patient interface.

In this way, the verification chip and the temperature sensor are electrically connected in series, allowing the temperature measurement function and the identify verification function to be coupled together, thereby reducing the complexity of the circuit.

If another consumable in the breathing system requires identity verification, a verification chip can also be added to its design, allowing the breathing system to perform identity verification for that consumable. Additionally, the breathing apparatus itself may be equipped with a verification chip to enable a higher-level device to confirm whether the breathing apparatus is authorized.

Furthermore, the breathing system may include a heating element electrically connected to the breathing apparatus, where the heating element is configured to regulate and maintain the temperature of the breathing gas passing through the breathing gas supply conduit.

The above arrangements of the verification chip(s) and the temperature sensor in the previous embodiment are also applicable to this embodiment; that is, the verification chip and the temperature sensor can be used in combination with the heating element.

It should be understood that for patients who require the use of respiratory aids, providing breathing gas with an appropriate temperature is beneficial. The heating element may regulate and maintain the temperature of the breathing gas passing through the breathing gas supply conduit, ensuring that the breathing gas provided to the patient is within a comfortable range.

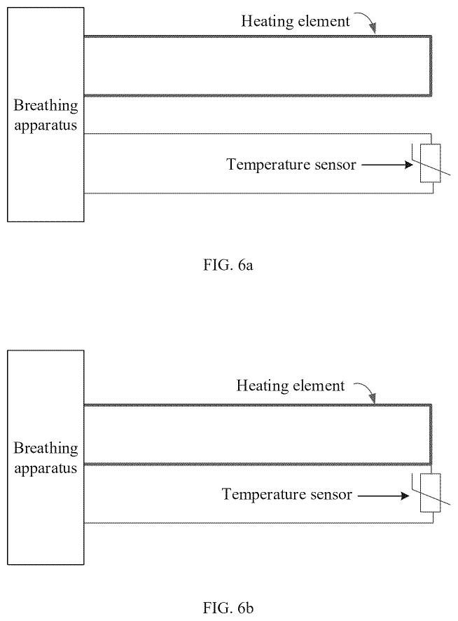

FIG. 6a is a schematic diagram of a circuit for a heating element and a temperature sensor according to an embodiment of the present disclosure. In FIG. 6a, the bold line represents the circuit for the heating element, or may be called a heating wire.

In an implementation, as show in FIG. 6a, the circuit for the electrical connection between the heating element and the breathing apparatus and the circuit for the electrical connection between the temperature sensor and the breathing apparatus are set up independently.

FIG. 6b is a schematic diagram of another circuit for a heating element and a temperature sensor according to an embodiment of the present disclosure. In FIG. 6b, the bold line represents the circuit for the heating element, or may be called a heating wire.

In this implementation, as shown in FIG. 6b, the circuit for the electrical connection between the heating element and the breathing apparatus and the circuit for the electrical connection between the temperature sensor and the breathing apparatus share one wire used for both of the circuits.

In one or more examples, the functions described may be implemented in hardware, software, firmware, or any combination thereof. For example, the functions may be implemented using one or more processors, such as application-specific integrated circuits (ASICs), field programmable gate arrays (FPGAs), or other equivalent integrated or discrete logic circuitry. Accordingly, the term “processor,” as used herein, may refer to any of the aforementioned structures or any other structure suitable for implementing the techniques described herein. Additionally, the techniques could be fully implemented in one or more circuits or logic elements.

Of course, the devices and components illustrated in the drawings may include elements not shown. The functions of the foreign object monitoring apparatus described in the specification can be realized through a circuit, which may include a subcircuit or a combination of subcircuits. In other words, the modules (the obtaining module and the determining module) described in the specification can be implemented as a subcircuit or a combination of subcircuits.

The foregoing detailed description has been presented for purposes of illustration and description. It is not intended to be exhaustive or to limit the subject matter claimed herein to the precise form(s) disclosed. Many modifications and variations are possible in light of the above teachings. The described embodiments were chosen in order to best explain the principles of the disclosed technology and its practical application to thereby enable others skilled in the art to best utilize the technology in various embodiments and with various modifications as are suited to the particular use contemplated. Those embodiments with various modifications are within the range and scope of the following claims.

What is claimed is:

1. A breathing system, comprising: a breathing apparatus, a breathing gas supply conduit, and a patient interface for a user;
wherein the breathing apparatus is operatively connected to one end of the breathing gas supply conduit, and the patient interface is operatively connected to the other end of the breathing gas supply conduit;
wherein the breathing apparatus is configured to provide breathing gas to the user through the breathing gas supply conduit and the patient interface; and
wherein at least one of the breathing gas supply conduit or the patient interface is equipped with a verification chip, and the verification chip is electrically connected to the breathing apparatus and used for identity verification of the at least one of the breathing gas supply conduit or the patient interface.

2. The breathing system according to claim 1, wherein when the breathing gas supply conduit is equipped with the verification chip, the verification chip of the breathing gas supply conduit carries first identity information of the breathing gas supply conduit, and the first identity information is used for the breathing apparatus to perform identity verification on the breathing gas supply conduit.

3. The breathing system according to claim 2, wherein the first identity information is a unique device identification (UDI) of the breathing gas supply conduit.

4. The breathing system according to claim 2, wherein the verification chip of the breathing gas supply conduit is positioned at the one end or the other end of the breathing gas supply conduit.

5. The breathing system according to claim 1, wherein when the patient interface is equipped with the verification chip, the verification chip of the patient interface carries second identity information of the patient interface, and the second identity information is used for the breathing apparatus to perform identity verification on the patient interface.

6. The breathing system according to claim 5, wherein the second identity information is a UDI of the patient interface.

7. The breathing system according to claim 5, wherein the patient interface comprises a cannula or a mask on which the verification chip of the patient interface is positioned.

8. The breathing system according to claim 1, further comprising a temperature sensor electrically connected to the breathing apparatus;
wherein the temperature sensor is configured to measure and report a temperature to the breathing apparatus; and
wherein the temperature sensor is positioned at the breathing gas supply conduit or the patient interface.

9. The breathing system according to claim 8, wherein when that the temperature sensor is positioned at the breathing gas supply conduit, the temperature sensor is positioned to be flushed with or recessed into a wall of the breathing gas supply conduit.

10. The breathing system according to claim 8, wherein when both of the verification chip and the temperature sensor are positioned at the breathing gas supply conduit or the patient interface, the verification chip and the temperature sensor are electrically connected to the breathing apparatus in series.

11. The breathing system according to claim 8, further comprising a heating element electrically connected to the breathing apparatus;
wherein the heating element is configured to regulate and maintain the temperature of the breathing gas passing through the breathing gas supply conduit.

12. The breathing system according to claim 11, wherein a circuit for an electrical connection between the heating element and the breathing apparatus and a circuit for an electrical connection between the temperature sensor and the breathing apparatus are set up independently.

13. The breathing system according to claim 11, wherein a circuit for an electrical connection between the heating element and the breathing apparatus and a circuit for an electrical connection between the temperature sensor and the breathing apparatus share one wire used for both of the circuits.
